(19) United States
(12) Patent Application Publication
(10) Pub. No.: US 2014/0228301 A1
(43) Pub. Date: Aug. 14, 2014

(54) RECOMBINANT HUMAN ALPHA-1-ANTITRYPSIN FOR THE TREATMENT OF INFLAMMATORY DISORDERS

(71) Applicant: rEVO Biologics, Inc., Framingham, MA (US)

(72) Inventors: Harry M. Meade, Newton, MA (US); Paul R. Bourdon, Southborough, MA (US)

(21) Appl. No.: 14/342,170

(22) PCT Filed: Dec. 19, 2012
(86) PCT No.: PCT/US2012/070638

§ 371 Date: Feb. 28, 2014

Related U.S. Application Data
(60) Provisional application No. 61/577,289, filed on Dec. 19, 2011.

(51) Int. Cl. A61K 38/57

(52) U.S. Cl. CPC A61K 38/57
USPC 514/20.9; 435/325; 435/353; 435/354; 800/14

(57) ABSTRACT

In one aspect, the disclosure relates to compositions comprising alpha-1-antitrypsin (AAT) and the production thereof. In some embodiments, the AAT is recombinantly produced. The disclosure also relates to methods of administering compositions comprising alpha-1-antitrypsin (AAT).

### RELATED APPLICATIONS

This application claims the benefit under 35 U.S.C. §119 of U.S. provisional application 61/577,289, filed Dec. 19, 2011, the entire contents of which is incorporated herein by reference.

### FIELD OF THE INVENTION

The present invention relates to the treatment of inflammatory conditions including asthma, emphysema, chronic obstructive pulmonary disease and chronic granulomatous lung disease i.e., sarcoid. In particular, the invention relates to treatment of these conditions using recombinant human alpha-1-antitrypsin.

### BACKGROUND OF THE INVENTION

Recombinant proteins provide effective therapies for many life-threatening diseases. The use of high expression level systems such as bacterial, yeast and insect cells for production of therapeutic protein is limited to small proteins without extensive post-translational modifications. Mammalian cell systems, while producing many of the needed post-translational modifications, are more expensive due to the complex, and, therefore, sophisticated culture systems that are required. Moreover, in these sophisticated cell culture methods reduced protein expression levels are often seen. Some of the limitations of mammalian cell culture systems have been overcome with the expression of recombinant proteins in transgenic mammals or avians. Proteins have been produced in mammary glands of various transgenic animals with expression levels suitable for cost effective production at the scale of hundreds of kilograms of protein per year.

### SUMMARY OF THE INVENTION

In one aspect the disclosure provides recombinant human alpha-1-antrypsin (AAT). In one aspect, recombinantly produced recombinant human alpha-1-antrypsin (AAT) is administered to a patient in need of AAT.

Unexpectedly, it was found that the administration of recombinant human alpha-1 antitrypsin (AAT) provides higher efficacy in the lung than a corresponding dosage of plasma derived AAT. Without being bound by any specific theory, it is believed that the glycosylation profile of recombinant AAT produced in the milk of transgenic goats provides an increased localization of the protein in the lung compared to that of plasma derived AAT.

In one aspect, the disclosure provides a composition comprising alpha-1-antitrypsin (AAT), wherein the AAT is recombinantly produced. In some embodiments, the AAT is produced in mammary epithelial cells of a non-human mammal. In some embodiments, the AAT is produced in a transgenic non-human mammal. In some embodiments, the non-human mammal is a goat, sheep, bison, camel, cow, pig, rabbit, buffalo, horse, rat, mouse or llama. In some embodiments, the non-human mammal is a goat. In some embodiments, the recombinantly produced AAT has enhanced deoxyhexose glycosylation compared to plasma-derived AAT. In some embodiments, the recombinantly produced AAT has been modified to increase the sialylation on the AAT-glyco-motifs.

In one aspect, the disclosure provides a composition comprising AAT wherein the AAT has a high level of deoxyhexose glycosylation. In one aspect, the disclosure provides a composition comprising AAT wherein the AAT has a high level of sialylation on the AAT-glyco-motifs. In one aspect, the disclosure provides a composition comprising AAT wherein the AAT has a high level of deoxyhexose glycosylation and a high level of sialylation on the AAT-glyco-motifs.

In some embodiments of any of the compositions of AAT described herein, the composition further comprises milk. In some embodiments of any of the compositions of AAT described herein, the composition further comprises a pharmaceutically acceptable carrier.

In one aspect, the disclosure provides mammary gland epithelial cells that produce the AAT of the compositions of any of the compositions described herein. In one aspect, the disclosure provides a transgenic non-human mammal comprising the mammary gland epithelial cells disclosed herein.

In one aspect, the disclosure provides methods of administering the AAT compositions disclosed herein to a subject in need thereof. In some embodiments, the subject has alpha-1-antitrypsin deficiency. In some embodiments, the subject has an inflammatory disorder. In some embodiments, the inflammatory disorder is emphysema. In some embodiments, the composition is administered at a dose of from 30 mg/kg to about 60 mg/kg AAT. In some embodiments, the composition is administered intravenously. In some embodiments the composition is administered by inhalation.

In one aspect, the disclosure provides a method of reducing elastase activity in the lung, the method comprising administering the AAT compositions disclosed herein to a subject in an amount sufficient to reduce elastase activity in the lung.

### BRIEF DESCRIPTION OF THE DRAWINGS

The drawings are exemplary and not required for enablement of the invention.

FIG. 1 shows Coomassie staining of rat broncholalveolar lavage (BAL) samples (FIG. 1A) and quantification of the staining of AAT harvested from BAL (FIGS. 1B and 1C). The samples are normalized based on albumin harvested from BAL.

FIG. 2 shows the level of GRO/CINC-1 ELISA (Growth-regulated gene product/cytokine-induced neutrophil chemoattractant) which is correlated with IL-8 and a model of inflammation in broncholalveolar lavage samples of rats treated with AAT.

FIG. 3 shows the elastase reactivity of control AAT (lanes 2-5) and broncholalveolar lavage harvested AAT (lanes 7-10). Binding to elastase is indicated by an increase in molecular weight of the AAT.

FIGS. 4 A and B shows the amount as assayed by SDS page of AAT harvested from the broncholalveolar lavage of rats which were administered a dose of 30 mg/kg of AAT.

FIG. 5 shows the amount as assayed by DOT blot analysis of AAT harvested from the broncholalveolar lavage of rats which were administered a dose of 30 mg/kg of AAT.

FIG. 6 shows the pharmacokinetics of AAT in rats exposed to 30 mg//kg of AAT.

FIG. 7 shows the relative level of AAT in blood vs. lung in rats that were administered 3 mg/kg AAT or 30 mg/kg AAT.

FIG. 8 shows the glycosylation pattern of recombinantly produced AAT.

FIG. 9 shows the glycosylation pattern of plasma-derived AAT.

FIGS. 10 A and B shows the amount as assayed by SDS page of AAT harvested from the broncholalveolar lavage of rats which were administered a dose of 3 mg/kg of AAT

FIG. 11 shows the amount as assayed by DOT blot analysis of AAT harvested from the broncholalveolar lavage of rats which were administered a dose of 3 mg/kg of AAT.

FIG. 12 shows the level of GRO/CINC-1 ELISA (Growth-regulated gene product/cytokine-induced neutrophil chemoattractant) which is correlated with IL-8 and a model of inflammation in broncholalveolar lavage samples of rats treated with AAT.

FIG. 13 shows the analysis of BAL from 30 mg/kg exposed rats.

FIG. 14 shows the analysis of BAL from 3 mg/kg exposed rats.

FIG. 15 shows the pharmacokinetics of 30 mg//kg rat lung study.

FIG. 16 shows the pharmacokinetics of 3 mg//kg rat lung study.

FIG. 17 shows anti-elastase activity. times

FIG. 18 shows the results and design of an experiment according to the methods provided herein.

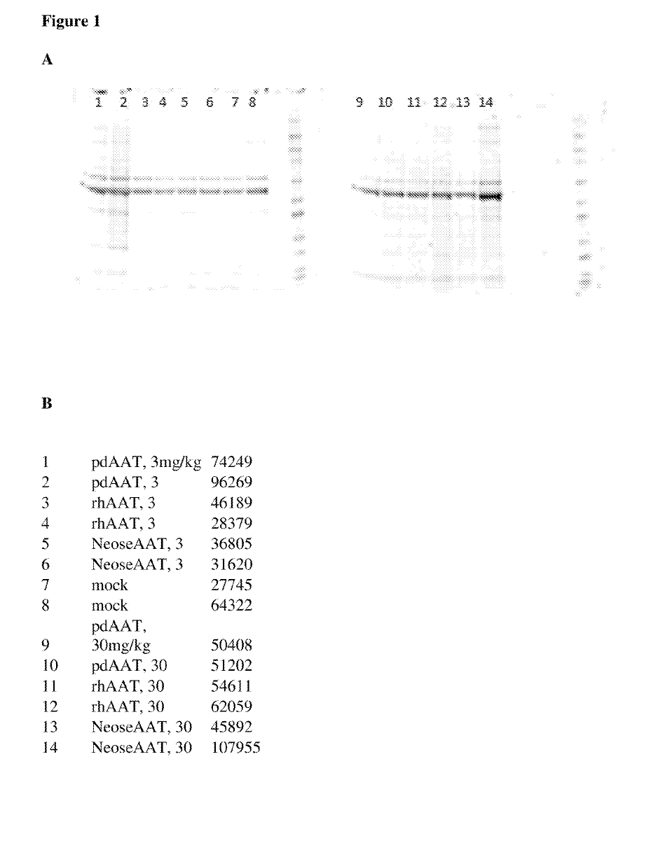

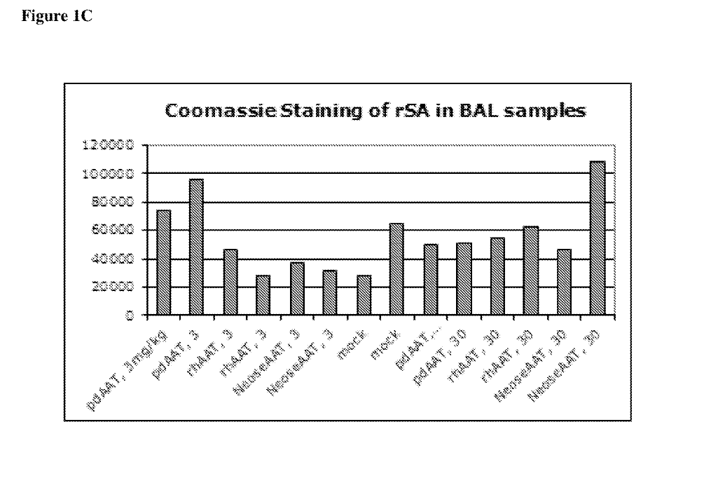

### DETAILED DESCRIPTION

In one aspect the disclosure provides compositions comprising recombinantly produced Alpha-1-antitrypsin (AAT) and methods of administering recombinantly produced AAT to a subject in need thereof.

Alpha-1-antitrypsin is a glycoprotein with a molecular weight of 53,000, as determined by sedimentation equilibrium centrifugation. The glycoprotein consists of a single polypeptide chain to which several oligosaccharide units (glyco-motifs) are covalently bonded. Human alpha 1-proteinase inhibitor has a role in controlling tissue destruction by endogenous serine proteinases. AAT is a suicide inhibitor that works by forming a stable tetrahedral intermediate with an enzyme, predominantly elastase, after binding. Completion of the cleavage reaction is dependent on hydrolysis of both the C-terminal peptide (leaving group) and the active site serine. In most cases, the first hydrolysis takes place and the enzyme is translocated across the beta sheet and “smashed”, disrupting the active site and rendering the enzyme inactive and unable to complete the second hydrolysis, which leaves the enzyme tethered to the AAT. If the second hydrolysis does occur, the AAT is released from the enzyme, minus its 36 amino acid peptide. Alpha-1-proteinase inhibitor inhibits human pancreatic and leukocyte elastases. See e.g., Pannell et al., Biochemistry 13, 5339 (1974); Johnson et al., Biochem Biophys Res Comm, 72 33 (1976); Del Mar et al., Biochem Biophys Res Commun, 88, 346 (1979); and Heimburger et al., Proc. Int. Res. Conf. Proteinase Inhibitors 1^{st}, 1-21 (1970).

A genetic deficiency of alpha-1-proteinase inhibitor, which accounts for 90% of the trypsin inhibitory capacity in blood plasma, has been shown to be associated with the premature development of pulmonary emphysema. The degradation of elastin associated with emphysema probably results from a local imbalance of elastolytic enzymes and the naturally occurring tissue and plasma proteinase inhibitors. Currently, subjects deficient in AAT are treated with therapeutic concentrates of alpha-1-antitrypsin prepared from the blood plasma of blood donors (plasma-derived AAT).

In one aspect, the disclosure provides methods of administering a composition comprising recombinantly produced AAT to a subject in need thereof. In one aspect, the disclosure provides methods of reducing elastase activity in the lung, the method comprising administering a composition comprising recombinantly produced AAT to a subject in an amount sufficient to reduce elastase activity in the lung.

Unexpectedly, it was found herein that recombinantly produced AAT (rhAAT) e.g., AAT produced in transgenic animals, upon administration is sequestered in the lung at higher levels than a corresponding dose of plasma derived AAT. As shown herein, rats were dosed with plasma-derived AAT, rhAAT or sialylated rhAAT, and in these rats rhAAT and sialylated rhAAT were found in the bronchial alveolar lavage (BAL) fluid at greater levels than plasma derived AAT. This sequestration into the lung was even more surprising because of the lower concentrations of rhAAT and sialylated rhAAT in the blood. For instance, as shown herein, two hours after administration, recombinantly produced AAT is present in BAL at a concentration approximately three times higher than the concentration of plasma-derived AAT. This is even more remarkable if taken into account that the concentration of recombinantly produced AAT in the blood at that same time is about six times lower than concentration of plasma-derived AAT. The concentration of recombinantly produced AAT in the blood is lower likely due to the higher clearance rate in the blood of recombinantly produced AAT compared to plasma-derived AAT. The effect of sequestration in the lung is even more pronounced when sialylated recombinant AAT is compared to plasma-derived AAT. Sialylated AAT has a lower clearance rate than unsialylated AAT and can therefore maintain a higher level of recombinant AAT in the system.

It is also shown herein that the recombinant AAT harvested from BAL can bind elastase and it thus remains effective in the treatment of lung disease. Furthermore, the recombinant AAT sequestered into the lung does not cause any more inflammation than found in a control experiment. Recombinantly produced AAT therefore has unexpected properties that make it well suited for the treatment of lung disorders and/or inflammatory disorders.

It should be appreciated that the AAT to be administered to a subject should generally be species-appropriate. In other words, if AAT is to be administered to a human, the AAT will likely be human AAT. However, AAT from other species may be administered (e.g., pig AAT administered to a human) as long as the AAT from a different species can still fulfill its biological role (e.g., bind human elastase) and does not cause an inappropriate immune response.

In one aspect, the disclosure provides compositions of recombinantly produced AAT, wherein the recombinantly produced AAT has enhanced deoxyhexose glycosylation compared to plasma-derived AAT. In one aspect, the disclosure provides compositions of recombinantly produced AAT, wherein the recombinantly produced AAT has been modified to increase the sialylation on the AAT-glyco-motifs.

Recombinantly produced AAT has the same amino acid sequence as plasma-derived AAT. However, recombinantly produced AAT has a glycosylation pattern that is different from (human) plasma-derived AAT, as shown in the experimental section. In some embodiments, the recombinant AAT is produced in non-human mammary epithelial cells. The recombinant AAT produced in non-human mammary epithelial cells has a glycosylation pattern that is determined inter alia by the prevalence and interaction of glycosylation enzymes present in these mammary epithelial cells.

While not being limited to a specific mechanism, it is assumed that recombinantly produced AAT is sequestered in the lung because it has a higher affinity than plasma-derived AAT for glyco-receptors present in the lung (receptors that bind the AAT glycoprotein and/or the glyco-motifs of the AAT glyco-protein). Again, while not being limited to a specific mechanism the small amount of exposed N-acetylglucosamine present on recombinant AAT, which can bind the mannose receptor present in the lung, may be responsible for the accumulation of recombinant AAT in the lung. Alternatively, or in addition, deoxyhexose, which is present in larger amounts in the glyco motifs of recombinantly produced AAT than in plasma-derived AAT, may be responsible for the sequestering in the lung.

In one aspect the disclosure provides a composition comprising AAT wherein the AAT has a high level of deoxyhexose glycosylation. In one aspect the disclosure provides a composition comprising AAT with a high level of sialylation on the AAT-glyco-motifs. In one aspect the disclosure provides a composition comprising AAT wherein the AAT has a high level of deoxyhexose glycosylation and a high level of sialylation on the AAT-glyco-motifs.

It should further be appreciated that AAT that has a glycosylation pattern that is the same as the glycosylation pattern of recombinantly produced AAT can also be used in the methods described herein. Thus, in some embodiments the disclosure provides compositions and methods for the administration of AAT that is not recombinantly produced, but that has the same glycosylation pattern as recombinantly produced AAT. Thus, in some embodiments, the disclosure provides compositions and methods of administration of AAT comprising exposed N-acetylglucosamine. In some embodiments, the disclosure provides compositions and methods of administration of AAT comprising a high level of deoxyhexose glycosylation. In some embodiments, a high level of deoxyhexose glycosylation as used herein refers to a level of deoxyhexose glycosylation that is 1.1 times or more, 1.2 times or more, 1.3 times or more, 1.5 times or more, 2 times or more, 5 times or more, 10 times or more, 50 times or more, or 100 times or more than the level of deoxyhexose glycosylation found in plasma-derived AAT. In some embodiments, a high level of deoxyhexose glycosylation as used herein refers to a population of AAT wherein at least 50%, at least 60%, at least 70%, at least 80%, at least 90% up to 100% of the glyco-motifs include a deoxyhexose moiety. In some embodiments, the disclosure provides compositions and methods of administration of AAT comprising a high level of sialylation on the ATT glyco-motifs. In some embodiments, a high level of sialylation on the ATT glyco-motifs as used herein refers to a population of AAT wherein at least 50%, at least 60%, at least 70%, at least 80%, at least 90% up to 100% of the glyco-motifs in a population of AAT are sialylated.

Methods of modifying the glycosylation motif of a glycoprotein such as AAT are known in the art. For instance, a plasma-derived or E. coli-produced AAT can be subjected to enzymatic treatment with one or more glycosylation enzymes to increase the amount of N-acetylglucosamine and/or deoxyhexose. For instance, treatment of AAT with neuraminidase followed by beta galactosidase may increase the amount of exposed N-acetylglucosamine.

### Non-Human Mammary Gland Epithelial Cells for the Production of AAT

In one aspect, the disclosure provides mammary gland epithelial cells that produce AAT. In one aspect, the disclosure provides a transgenic non-human mammal that produces AAT. In one aspect, the disclosure relates to mammalian mammary epithelial cells that produce AAT. Methods are provided herein for producing glycosylated AAT in mammalian mammary epithelial cells. This can be accomplished in cell culture by culturing mammary epithelial cell (in vitro or ex vivo). This can also be accomplished in a transgenic animal (in vivo).

In some embodiments, the mammalian mammary gland epithelial cells are in a transgenic animal. In some embodiments, the mammalian mammary gland epithelial cells have been engineered to express AAT in the milk of a transgenic animal, such as a mouse or goat. To accomplish this, the expression of the gene(s) encoding the recombinant protein can be, for example, under the control of the goat β-casein regulatory elements. Expression of recombinant proteins in both mice and goat milk has been established previously (see, e.g., US Patent Application US-2008-0118501-A1). In some embodiments, the expression is optimized for individual mammary duct epithelial cells that produce milk proteins.

Transgenic animals capable of producing recombinant AAT can be generated according to methods known in the art (see, e.g., U.S. Pat. No. 5,945,577 and US Patent Application US-2008-0118501-A1) such methods are incorporated herein. Animals suitable for transgenic expression, include, but are not limited to goat, sheep, bison, camel, cow, pig, rabbit, buffalo, horse, rat, mouse or llama. Suitable animals also include bovine, caprine, ovine and porcine, which relate to various species of cows, goats, sheep and pigs (or swine), respectively. Suitable animals also include ungulates. As used herein, “ungulate” is of or relating to a hoofed typically herbivorous quadruped mammal, including, without limitation, sheep, swine, goats, cattle and horses. Suitable animals also include dairy animals, such as goats and cattle, or mice. In some embodiments, the animal suitable for transgenic expression is a goat.

In one embodiment, transgenic animals are generated by generation of primary cells comprising a construct of interest followed by nuclear transfer of primary cell nuclei into enucleated oocytes. Primary cells comprising a construct of interest are produced by injecting or transfecting primary cells with a single construct comprising the coding sequence of a protein of interest, e.g., AAT. These cells are then expanded and characterized to assess transgene copy number, transgene structural integrity and chromosomal integration site. Cells with desired transgene copy number, transgene structural integrity and chromosomal integration sites are then used for nuclear transfer to produce transgenic animals. As used herein, “nuclear transfer” refers to a method of cloning wherein the nucleus from a donor cell is transplanted into an enucleated oocyte.

Coding sequences for AAT to be expressed in mammalian mammary epithelial cells can be obtained by screening libraries of genomic material or reverse-translated messenger RNA derived from the animal of choice (such as humans, cattle or mice), from sequence databases such as NCBI, Genbank, or by obtaining the sequences by using methods known in the art, e.g. peptide mapping. The sequences can be cloned into an appropriate plasmid vector and amplified in a suitable host organism, like E. coli. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. After amplification of the vector, the DNA construct can be excised, purified from the remains of the vector and introduced into expression vectors that can be used to produce transgenic animals. The transgenic animals will have the desired transgenic protein integrated into their genome.

A DNA sequence which is suitable for directing production to the milk of transgenic animals can carry a 5′-promoter region derived from a naturally-derived milk protein. This promoter is consequently under the control of hormonal and tissue-specific factors and is most active in lactating mammary tissue. In some embodiments the promoter used is a milk-specific promoter. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, κ-casein promoter, whey acidic protein (WAP) promoter (Gorton et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV). In some embodiments the promoter is a caprine beta casein promoter.

The promoter can be operably linked to a DNA sequence directing the production of a protein leader sequence which directs the secretion of the transgenic protein across the mammary epithelium into the milk. As used herein, a coding sequence and regulatory sequences (e.g., a promoter) are said to be “operably joined” or “operably linked” when they are linked in such a way as to place the expression or transcription of the coding sequence under the influence or control of the regulatory sequences. As used herein, a “leader sequence” or “signal sequence” is a nucleic acid sequence that encodes a protein secretory signal, and, when operably linked to a downstream nucleic acid molecule encoding a transgenic protein, directs secretion. The leader sequence may be the native human leader sequence, an artificially-derived leader, or may be obtained from the same gene as the promoter used to direct transcription of the transgene coding sequence, or from another protein that is normally secreted from a cell, such as a mammalian mammary epithelial cell. In some embodiments a 3′-sequence, which can be derived from a naturally secreted milk protein, can be added to improve stability of mRNA.

In some embodiments, to produce primary cell lines containing a construct (e.g., encoding AAT) for use in producing transgenic goats by nuclear transfer, the constructs can be transfected into primary goat skin epithelial cells, which are expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Cloning will result in a multiplicity of transgenic animals—each capable of producing an AAT or other gene construct of interest. The production methods include the use of the cloned animals and the offspring of those animals. Cloning also encompasses the nuclear transfer of fetuses, nuclear transfer, tissue and organ transplantation and the creation of chimeric offspring. One step of the cloning process comprises transferring the genome of a cell, e.g., a primary cell that contains the transgene of interest into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal. Suitable mammalian sources for oocytes include goats, sheep, cows, pigs, rabbits, guinea pigs, mice, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Thus, in one aspect the disclosure provides mammary gland epithelial cells that produce the AAT disclosed herein. In some embodiments, the mammary epithelial cells above are in a transgenic non-human mammal. In some embodiments, the transgenic non-human mammal is a goat.

### Transgenic Animals

In one aspect, the present disclosure also provides a method of generating a genetically engineered or transgenic mammal, by which a desired gene is inserted in the pronucleus of a pre-implantation enbryo. The genetic material integrates into the genome and the resulting animal carries the genetic material in its genome. In this case the transgene provides the genetic information for expression of the recombinant AAT into the milk of the lactating female.

In one aspect, the present disclosure also provides a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

In one aspect, the present disclosure also provides mammals obtained according to the methods provided herein, and the offspring of those mammals. In some embodiments, the present disclosure is used for generating caprines or bovines, but the methods can be used with any non-human mammalian species. The present disclosure further provides for the use of nuclear transfer fetuses and nuclear transfer and chimeric offspring in the area of cell, tissue and organ transplantation.

Suitable mammalian sources for embryos and oocytes include goats, sheep, cows, pigs, rabbits, guinea pigs, mice, hamsters, rats, primates, etc., Preferably, in some embodiments, the oocytes are obtained from ungulates, and most preferably, in some embodiments, goats or cattle. Methods for isolation of oocytes are well known in the art. Essentially, oocytes are isolated from the ovaries or reproductive tract of a mammal, e.g., goat. A readily available source of ungulate oocytes is from hormonally induced female animals.

For the successful use of techniques such as genetic engineering, nuclear transfer and cloning, oocytes may preferably be matured in vivo before these cells may be used as recipient cells for nuclear transfer, and before they are fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins optimized for use as a therapeutic in humans in terms of their glycan profile. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields). The current invention enables the use of transgenic production of biopharmaceuticals, transgenic proteins, plasma proteins, and other molecules of interest in the milk or other bodily fluid (e.g., urine or blood) of transgenic animals transgenic for a desired gene that then optimizes the glycosylation profile of those molecules.

A DNA sequence which is suitable for directing production to the milk of transgenic animals carries a 5′-promoter region derived from a naturally-derived milk protein and is consequently under the control of hormonal and tissue-specific factors. Such a promoter should therefore be most active in lactating mammary tissue. According to the current invention the promoter so utilized are followed by a DNA sequence directing the production of a protein leader sequence which would direct the secretion of the transgenic protein across the mammary epithelium into the milk. At the other end of the transgenic protein construct a suitable 3′-sequence, preferably also derived from a naturally secreted milk protein, may be added to improve stability of mRNA. Examples of suitable control sequences for the production of proteins in the milk of transgenic animals are those from the caprine beta casein promoter.

The production of transgenic animals can now be performed using a variety including micro-injection and nuclear transfer techniques.

### Methods of Production of AAT

In one aspect, the disclosure provides methods for production of AAT. In one aspect, the disclosure provides a method for producing AAT comprising expressing the AAT in mammary gland epithelial cells of a non-human mammal. In some embodiments, the mammary gland epithelial cells are in culture and are transfected with a nucleic acid that comprises a sequence that encodes the AAT. In some embodiments, the mammary gland epithelial cells are in a non-human mammal engineered to express a nucleic acid that comprises a sequence that encodes AAT in its mammary gland. In some embodiments, the mammary gland epithelial cells are goat, sheep, bison, camel, cow, pig, rabbit, buffalo, horse, rat, mouse or llama mammary gland epithelial cells. In some embodiments, the mammary gland epithelial cells are goat mammary gland epithelial cells.

In one aspect the disclosure provides mammary gland epithelial cells that express AAT as disclosed herein.

In one aspect the disclosure provides a transgenic non-human mammal comprising mammary gland epithelial cells that express AAT as disclosed herein.

In another aspect the disclosure provides a method for the production of a transgenic AAT the process comprising expressing in the milk of a transgenic non-human mammal AAT encoded by a nucleic acid construct. In some embodiments, the method for producing AAT comprises:

(a) transfecting non-human mammalian cells with a transgene DNA construct encoding AAT;

(b) selecting cells in which said AAT transgene DNA construct has been inserted into the genome of the cells; and

(c) performing a first nuclear transfer procedure to generate a non-human transgenic mammal heterozygous for AAT and that can express it in its milk.

In another aspect, the disclosure provides a method of:

(a) providing a non-human transgenic mammal engineered to express AAT;

(b) expressing AAT in the milk of the non-human transgenic mammal; and

(c) isolating AAT in the milk.

One of the tools used to predict the quantity and quality of the recombinant protein expressed in the mammary gland is through the induction of lactation (Ebert KM, 1994). Induced lactation allows for the expression and analysis of protein from the early stage of transgenic production rather than from the first natural lactation resulting from pregnancy, which is at least a year later. Induction of lactation can be done either hormonally or manually.

In some embodiments, the compositions of AAT provided herein further comprise milk. In some embodiments, the methods provided herein include a step of isolating AAT from the milk of a transgenic animal. Methods for isolating proteins from the milk of transgenic mammals are known in the art and are described for instance in Pollock et al., Journal of Immunological Methods, Volume 231, Issues 1-2, 10 Dec. 1999, Pages 147-157. In some embodiments, the methods provided herein include a step of purifying the expressed AAT.

In one aspect the disclosure provides a method for the production of AAT comprising expressing in the milk of a transgenic non-human mammal AAT by a nucleic acid construct. In one embodiment the mammalian mammary epithelial cells are of a non-human mammal engineered to express the AAT in its milk. In some embodiments, the mammalian mammary epithelial cells are mammalian mammary epithelial cells in culture.

In another embodiment the method comprises:

(a) providing a non-human transgenic mammal engineered to express AAT,

(b) expressing the AAT in the milk of the non-human transgenic mammal;

(c) isolating the AAT expressed in the milk.

In yet another embodiment the method comprises: producing AAT in mammary gland epithelial cells such that the AAT has a high level of deoxyhexose. In some embodiments, this method is performed in vitro. In other embodiments, this method is performed in vivo, e.g., in the mammary gland of a transgenic goat.

In some embodiments the methods above further comprise steps for inducing lactation. In some embodiments the methods further comprise additional isolation and/or purification steps. In some embodiments the methods further comprise steps for comparing the glycosylation pattern of recombinantly produced AAT with plasma-derived AAT. In further embodiments, the methods further comprise steps for comparing the glycosylation pattern of recombinantly produced AAT to plasma-derived AAT.

In some embodiments, the methods further include a step of sialylating the glycopeptides of AAT.

In some embodiments, the method further comprises comparing the percentage of deoxyhexose glycosylation present in a population of recombinantly produced AAT to the percentage of deoxyhexose glycosylation in a population of plasma-derived AAT. Experimental techniques for assessing the glycosylation pattern of AAT can be any of those known to those of ordinary skill in the art or as provided herein, such as below in the Examples. Such methods include, e.g., liquid chromatography mass spectrometry, tandem mass spectrometry, and Western blot analysis.

Recombinantly produced AAT can be obtained, in some embodiments, by collecting the AAT from the milk of a transgenic animal produced as provided herein or from an offspring of said transgenic animal. In some embodiments the AAT produced by the transgenic mammal is produced at a level of at least 1 gram per liter of milk produced. In some embodiments, the goats expressing rhAAt are produced using microinjection methods.

### Methods of Treatment, Pharmaceutical Compositions, Dosage, and Administration

In one aspect the disclosure provides method of administering a composition of AAT to a subject in need thereof. In some embodiments the AAT is recombinantly produced. In some embodiments, the AAT is produced in non-human mammary epithelial cells. In some embodiments, the AAT has a high level of deoxyhexose glycosylation. In some embodiments, the AAT has a high level of sialylation on the AAT-glyco-motifs. In some embodiments, the AAT has a high level of deoxyhexose glycosylation and a high level of sialylation on the ATT-glyco-motifs.

In one aspect the disclosure provides methods of administering a composition of AAT to a subject in need thereof. In some embodiment, the subject has alpha-1-antitrypsin deficiency. In some embodiments, the subject has an inflammatory disorder or autoimmune disorder. In some embodiment, the inflammatory disorder is emphysema. In some embodiment, the inflammatory disorder or immune disorders include but are not limited, to adult respiratory distress syndrome, arteriosclerosis, asthma, atherosclerosis, cholecystitis, cirrhosis, Crohn's disease, diabetes mellitus, emphysema, hypereosinophilia, inflammation, irritable bowel syndrome, multiple sclerosis, myasthenia gravis, myocardial or pericardial inflammation, osteoarthritis, osteoporosis, pancreatitis, rheumatoid arthritis, scleroderma, colitis, systemic lupus erythematosus, lupus nephritis, diabetes mellitus, inflammatory bowel disease, celiac disease, an autoimmune thyroid disease, Addison's disease, Sjogren's syndrome, Sydenham's chorea, Takayasu's arteritis, Wegener's granulomatosis, autoimmune gastritis, autoimmune hepatitis, cutaneous autoimmune diseases, autoimmune dilated cardiomyopathy, multiple sclerosis, myocarditis, myasthenia gravis, pernicious anemia, polymyalgia, psoriasis, rapidly progressive glomerulonephritis, rheumatoid arthritis, ulcerative colitis, vasculitis, autoimmune diseases of the muscle, autoimmune diseases of the testis, autoimmune diseases of the ovary and autoimmune diseases of the eye, acne vulgari, asthma, autoimmune diseases, celiac disease, chronic prostatitis, glomerulonephritis, hypersensitivities, inflammatory bowel diseases, pelvic inflammatory disease, peperfusion injury, rheumatoid arthritis, sarcoidosis, transplant rejection, vasculitis, and interstitial cystitis.

In one aspect, the disclosure provides methods of reducing elastase activity in the lung, comprising administering a composition of AAT to a subject in an amount sufficient to reduce elastase activity in the lung.

In one aspect, the disclosure provides pharmaceutical compositions which comprise AAT and a pharmaceutically acceptable vehicle, diluent or carrier. In some embodiments, the compositions provided herein comprise milk.

In one aspect, the disclosure provides a method of treating a subject, comprising administering to a subject a composition provided in an amount effective to treat a disease the subject has or is at risk of having. In one embodiment the subject is a human. In another embodiment the subject is a non-human animal, e.g., a dog, cat, horse, cow, pig, sheep, goat or primate.

According to embodiments that involve administering to a subject in need of treatment a therapeutically effective amount of AAT as provided herein, “therapeutically effective” or “an amount effective to treat” denotes the amount of AAT or of a composition needed to inhibit or reverse a disease condition alleviate or prevent symptom thereof (e.g., to treat the inflammation). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. Efficacy, for example, can be measured by a decrease in inflammation or symptom thereof. A pharmaceutically effective amount, therefore, is an amount that is deemed by the clinician to be toxicologically tolerable, yet efficacious.

Dosage may be adjusted appropriately to achieve desired drug (e.g., AAT) levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of AAT. Appropriate systemic levels can be determined by, for example, measurement of the patient's peak or sustained plasma level of the drug. “Dose” and “dosage” are used interchangeably herein.

In some embodiments, the amount of AAT or pharmaceutical composition administered to a subject is 50 to 500 mg/kg, 100 to 400 mg/kg, or 200 to 300 mg/kg per week. In one embodiment the amount of AAT or pharmaceutical composition administered to a subject is 250 mg/kg per week. In some embodiments, an initial dose of 400 mg/kg is administered a subject the first week, followed by administration of 250 mg/kg to the subject in subsequent weeks. In some embodiments the administration rate is less than 10 mg/min. In some embodiments, administration of the AAT or pharmaceutical composition to a subject occurs at least one hour prior to treatment with another therapeutic agent. In some embodiments, a pre-treatment is administered prior to administration of AAT.

In some embodiments, the AAT or composition thereof is administered at a dose of 30 mg/kg to about 60 mg/kg.

In some embodiments the compositions provided are employed for in vivo applications. Depending on the intended mode of administration in vivo the compositions used may be in the dosage forms of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluents or carriers are amounts which are effective to obtain a pharmaceutically acceptable formulation in terms of solubility of components, biological activity, etc. In some embodiments the compositions provided herein are sterile.

Administration during in vivo treatment may be by any number of routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the disorder to be treated. For example, the compositions or AAT herein may be administered to a subject via oral, parenteral or topical administration. In one embodiment, the compositions or AAT herein are administered by intravenous infusion.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The component or components may be chemically modified so that oral delivery of the AAT is efficacious. Generally, the chemical modification contemplated is the attachment of at least one molecule to the AAT, where said molecule permits (a) inhibition of proteolysis; and (b) uptake into the blood stream from the stomach or intestine. Also desired is the increase in overall stability of the AAT and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that can be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the AAT or by release of the biologically active material beyond the stomach environment, such as in the intestine.

For buccal administration, the compositions may take the form of tablets or lozenges formulated in conventional manner.

For administration by inhalation, the compositions for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch.

Also contemplated herein is pulmonary delivery. The compositions can be delivered to the lungs of a mammal while inhaling and traverses across the lung epithelial lining to the blood stream. Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition disclosed herein is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

The compositions may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference. The AAT and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the disclosure contain an effective amount of the AAT and, optionally, other therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

The therapeutic agent(s), including specifically but not limited to the AAT may be provided in particles. Particles as used herein include nano or microparticles (or in some instances larger) which can consist in whole or in part of the AAT or other therapeutic agents administered with the AAT. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the AAT in a solution or in a semi-solid state. The particles may be of virtually any shape.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Pharmacokinetic Study of Alpha 1-Antitrypsin (AAT) in Rats

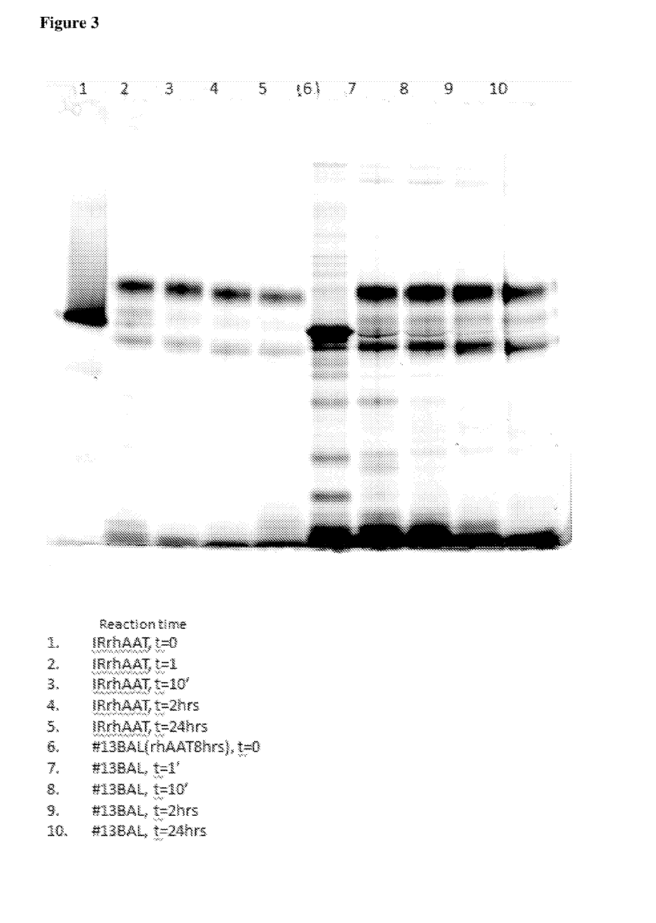

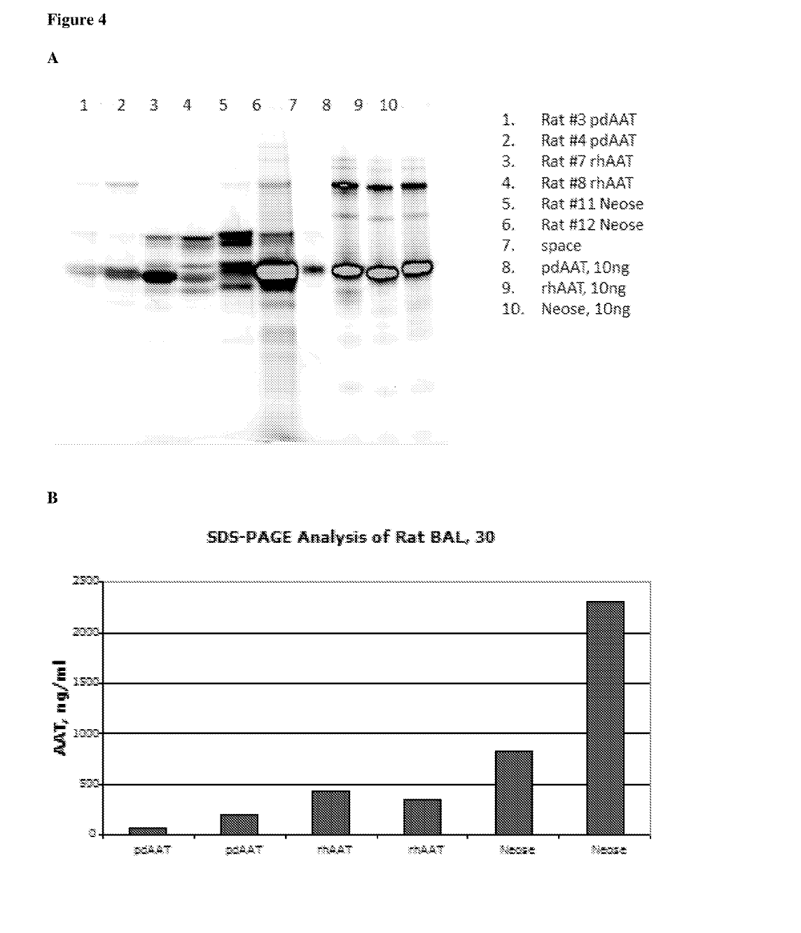

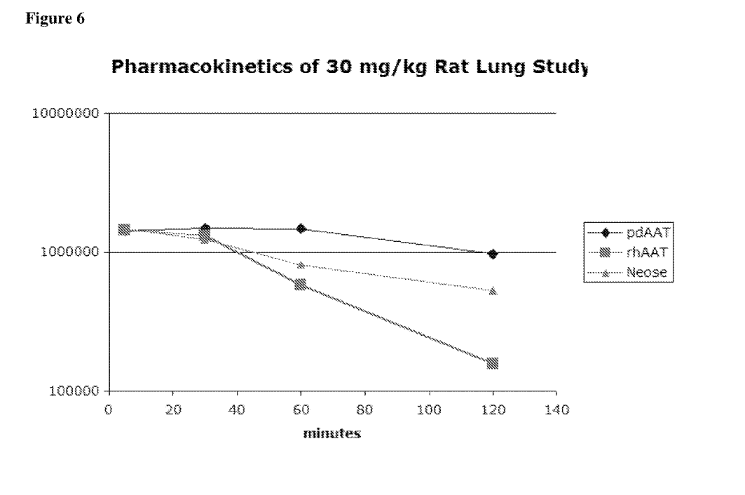

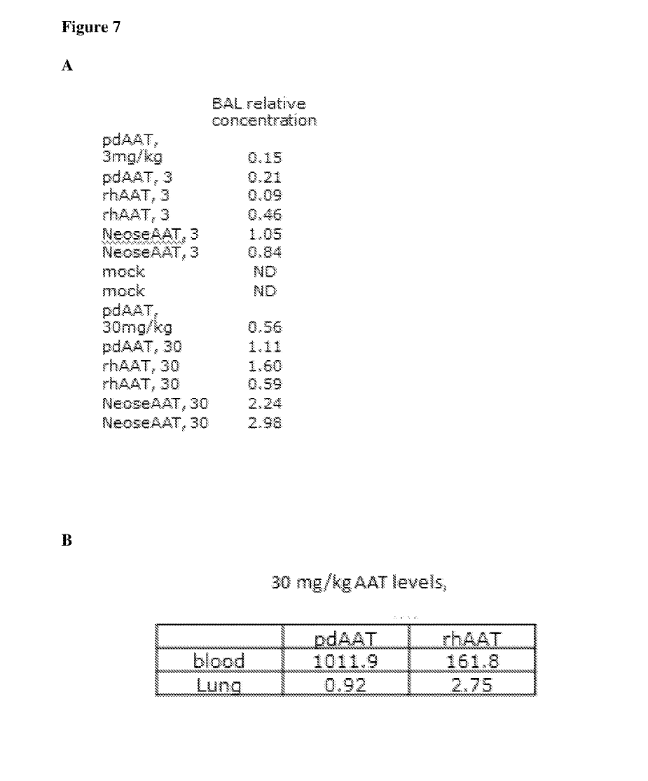

Plasma-derived (pdAAT), recombinantly produced (rhAAT) and sialylated recombinantly produced AAT (Neose) (AAT) were labeled with infrared dye and injected into rats at 3 and 30 mg/kg. Blood concentrations were followed by dot-blot and infrared scan analysis of samples taken over two hours at which point the animals were sacrificed and bronchial-alveolar lavage (BAL) fluid was collected. BAL samples were run on SDS-PAGE and the concentration of AAT was quantitated by infrared analysis and comparison to a standard curve of the starting material also run on SDS-PAGE. The data presented herein demonstrate that while the level of recombinant AAT was decreased in the blood compared to the plasma derived, the concentrations in the lung were comparable (See FIGS. 4, 5 and 7). Also, the sialylation of the recombinant AAT (“neose”) greatly improved the PK profile of rhAAT (See FIG. 6). The sialylation improves bioavailability but does not seem to interfere with the ability of the protein to be sequestered by the lung (See e.g., FIG. 7). Approximately twice as much sialylated rhAAT was observed in the BAL as in the pdAAT treated rats (See FIGS. 4, 5 and 7). Activity of the BAL AAT was assessed by the addition of human neutrophil elastase to the samples and the observation of a shift of the MW of AAT in both the complexed (82 kD) and cleaved (47 kD) form on SDS-PAGE (See FIG. 3).

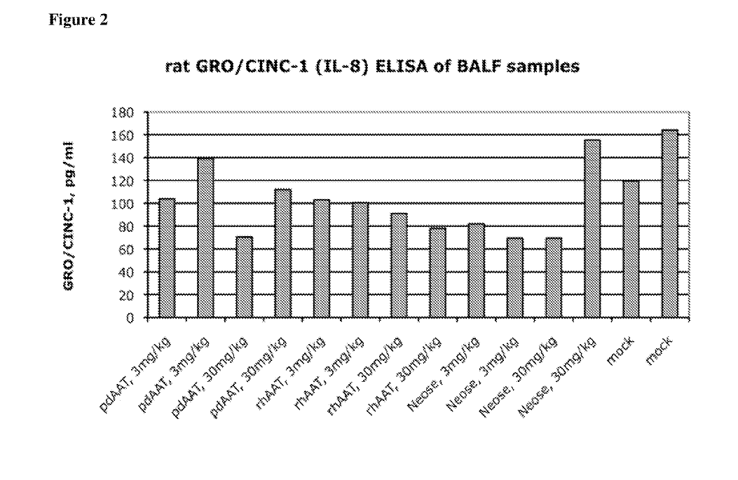

BAL samples were also run in an ELISA for rat GRO/CINC-1, an analog for human IL-8, to determine whether there was activation of the immune system by the recombinant AAT or sialylated recombinant AAT. Samples were diluted 1/10 in dilution buffer and compared to a standard curve. A GRO/CINC-1 assay was used to determine the extent of inflammation in the lungs (See FIG. 2). Low levels of IL-8, and thus low levels of inflammation, were observed for all samples.

Recombinant AAT and sialylated recombinant AAT are sequestered into the lung. A study was performed at two doses of AAT, 3 and 30 mg/kg and with plasma derived, recombinant and sialylated recombinant AAT. Two rats were included as mock controls to test for AAT activity in the BAL of an untreated animal. Each group included two rats. Injection was iv tail vein and blood samples were taken at 0, 5 30, 60 and 120 minutes when the rats were sacrificed and bronchial alveolar lavage fluid was collected by washing the lungs with 5 ml of PBS (See FIGS. 6 and 7).

Prior to the study, sialylated (Neose) recombinant AAT was generated by dialyzing recombinant AAT into HBS and treating for one hour with 50 mU of sialyltransferase 3 (ST3gal3) in 5 mM CMP-Nan. Sialylation of the terminal galactose was evaluated by an acidic shift on an IEF gel to a position very close to plasma derived AAT. All samples were labeled with IR800Dye CW, a NHS derivative of the infrared dye with absorption at 800 nm. Products were evaluated on SDS-PAGE and by anti-elastase activity assay (See FIG. 3). To determine whether the AAT in the BAL fluid was active, samples were mixed with 1 microgram of human neutrophil elastase, or AAT activity buffer, and run on SDS-PAGE. Lanes 1-5 shows the ability of rhAAT to bind elastase in vitro while lanes 7-10 show the ability to bind elastase after harvest from BAL.

Rat samples were assayed by diluting two microliters of serum into 200 microl of PBS and loading the samples on a piece of Protran 83 nitrocelullose with a 96 well vacuum manifold.

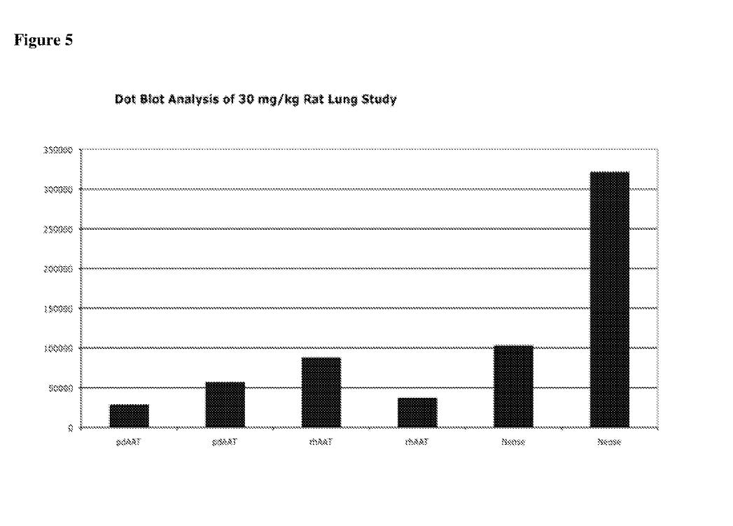

The filter was then scanned on an Odyssey infrared scanner at 800 nm. A grid was applied to the scan and integrated. BAL samples were also evaluated by SDS-PAGE. The presence of AAT in the lung was quantitated by integration of the bands at about the size of the monomer and above. The larger bands are different forms of labeled AAT including complexation with enzymes (See FIGS. 4 and 5).

### Results

Pharmacokinetic profiles showed that pdAAT has the slowest clearance and recombinant AAT the fastest clearance while sialylation (Neose) greatly reduced the clearance rate of rhAAT (See FIG. 6).

At 3 mg/kg the rats had detectable quantities of AAT in their BAL fluid samples. SDS-PAGE analysis of the samples demonstrated all forms could get into the lungs with the Neose treated AAT rat samples had more AAT in the lung than the plasma derived. rhAAT was detectable in the lung even with low levels in the blood (See FIGS. 6 and 7).

At 30 mg/kg, the level of recombinant AAT in BAL was actually three times greater than the plasma derived and sialylated recombinant AAT was more than 10 times the concentration of pdAAT.

In order to determine if the AAT observed in the lung samples (i.e., BAL) was active, one microgram of human neutrophil elastase was mixed with a rat sample and run on SDS-PAGE. All monomer disappeared and moved into one of three bands, slightly smaller, slightly larger and at approximately 80 kD, the expected size of an AAT:elastase complex. This was also observed when the starting material was mixed with elastase. A time course of this experiment demonstrated that the reaction was complete by one minute and the amount of each of the three bands did not change over 36 minutes.

The immunological state of the rat lung samples was examined by assaying for GRO/CINC-1, the rat analog of IL-8. Again, there was about 2-fold variation but levels were low in the range of 75 to 160 pg/ml.

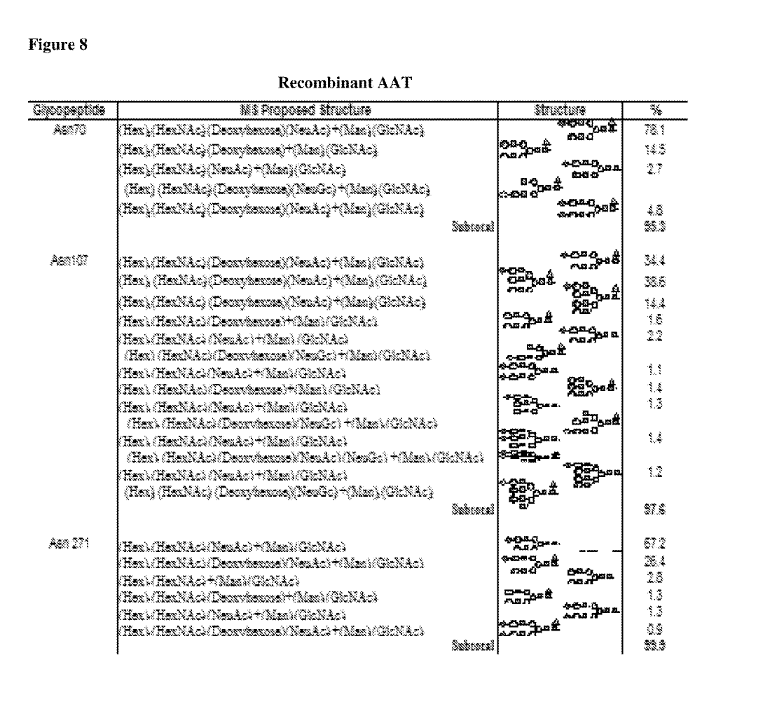

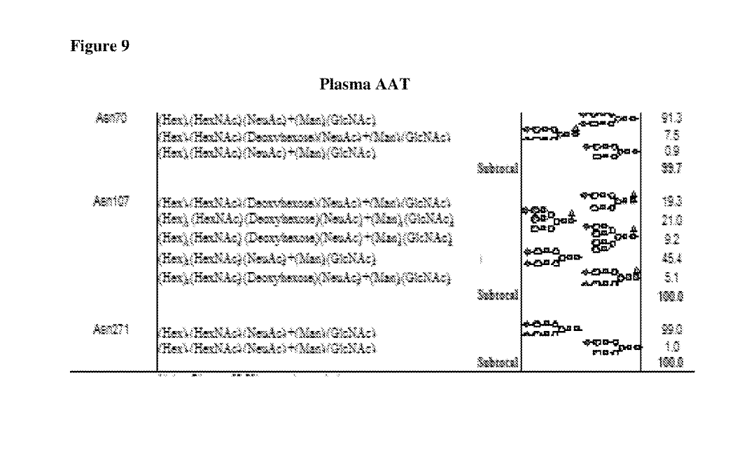

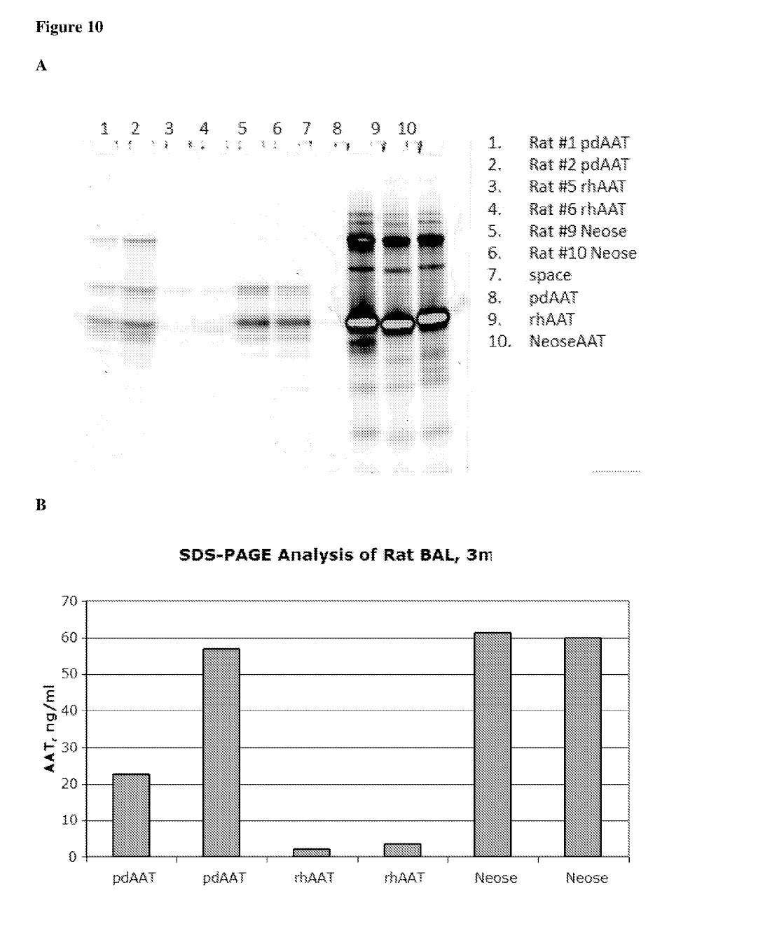

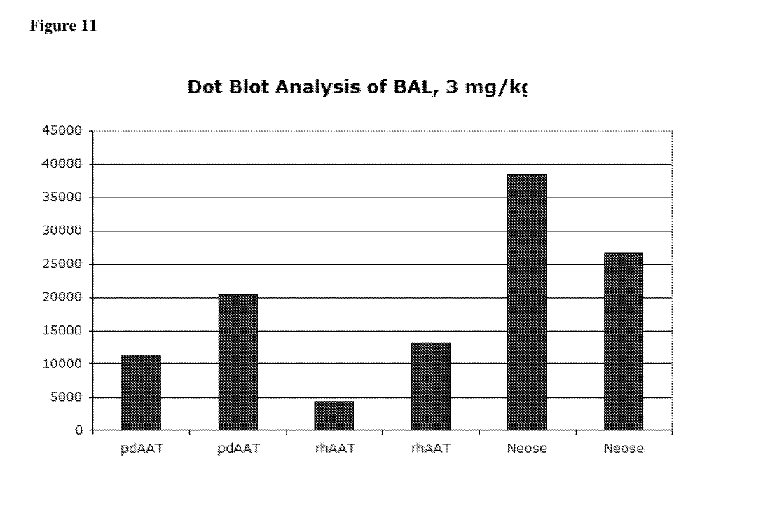

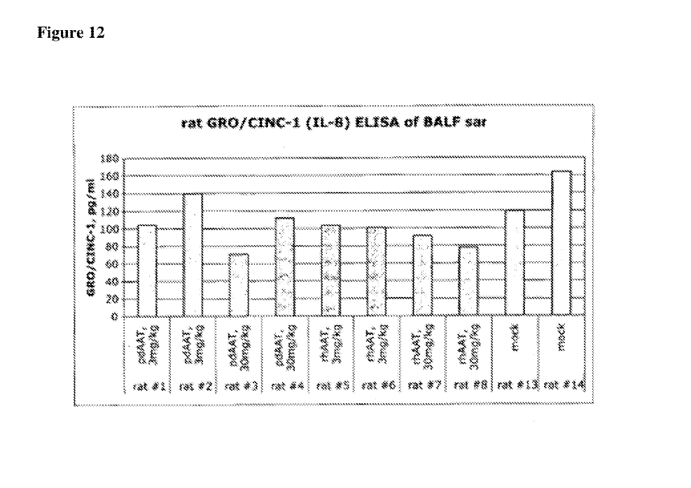

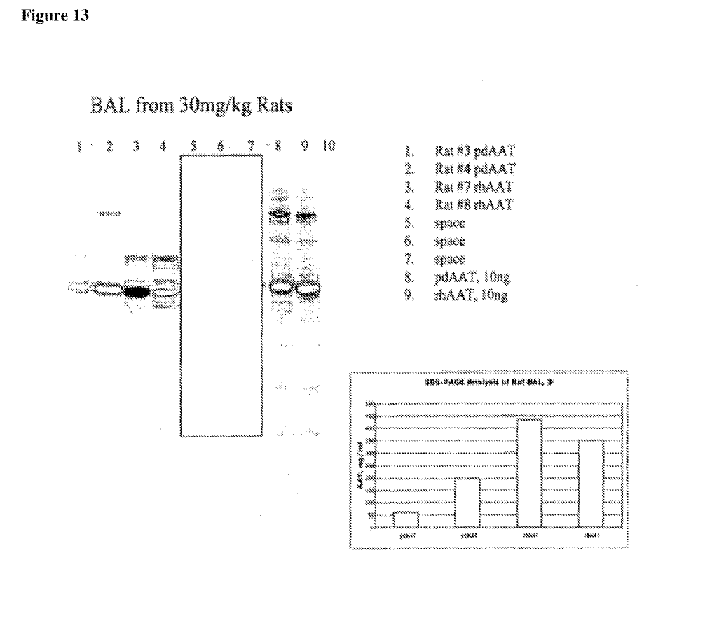

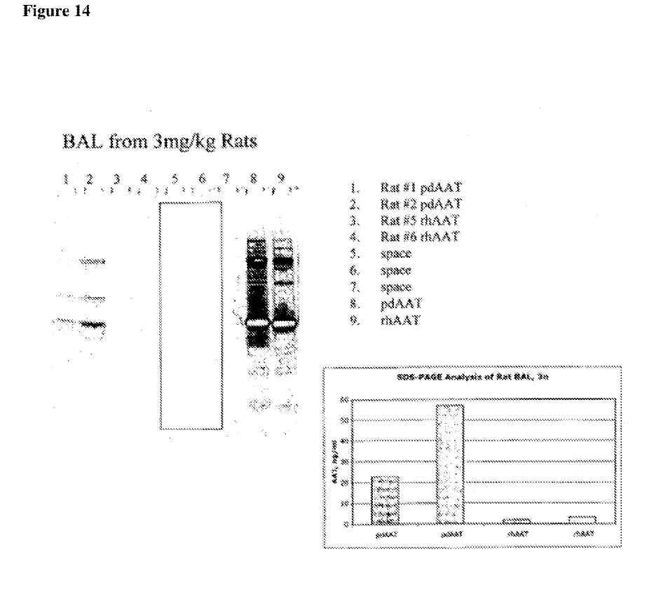

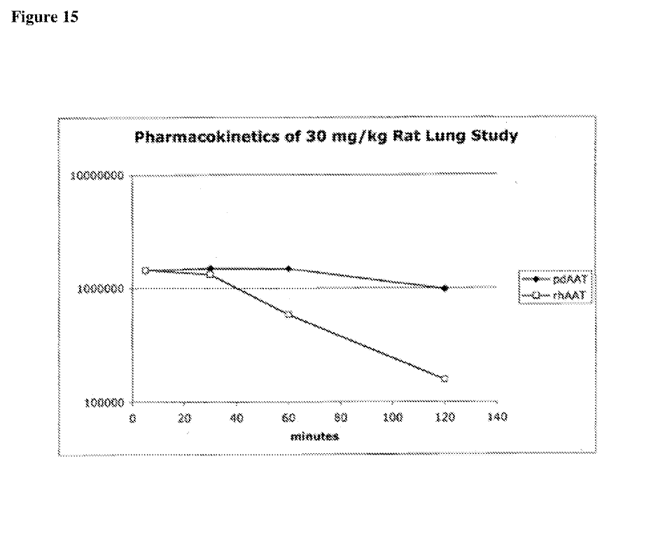

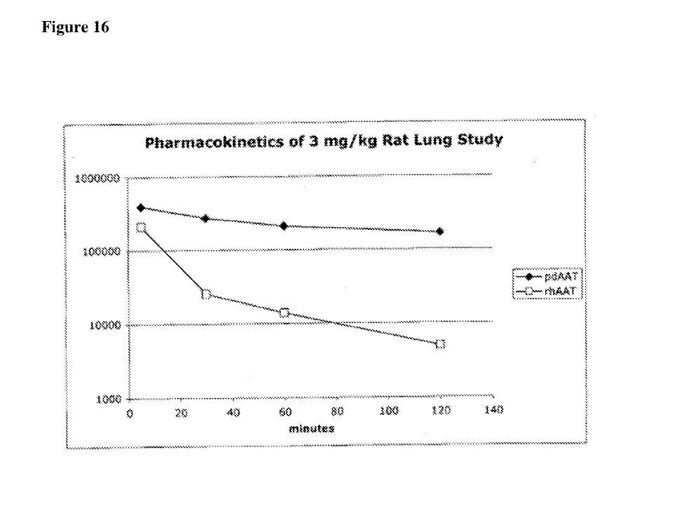

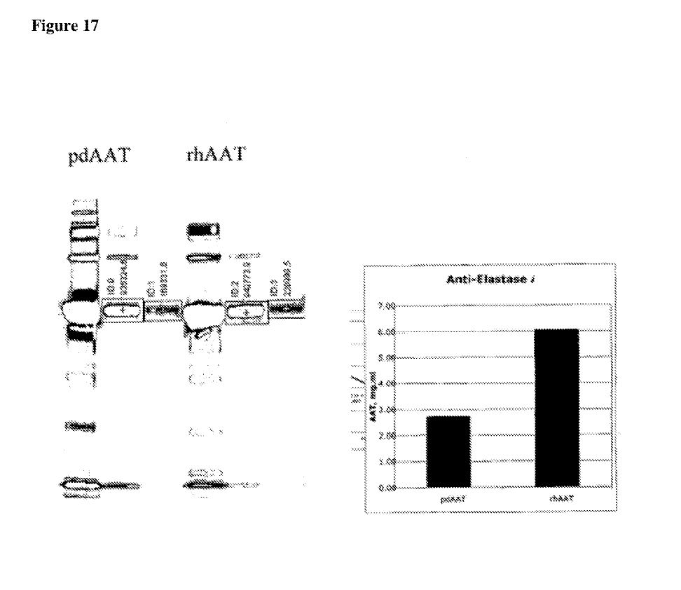

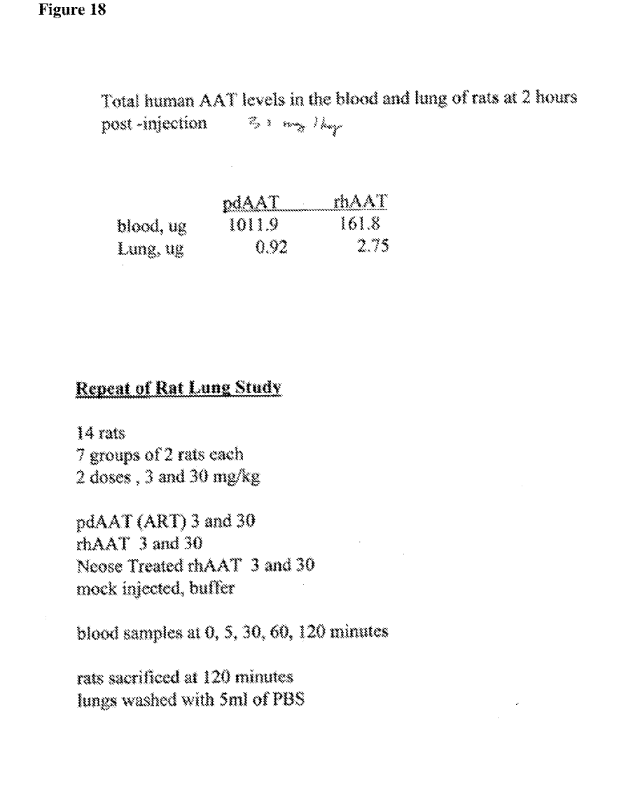

The glycosylation pattern of recombinant AAT and plasma AAT was also evaluated. The main difference is the lower level of deoxyhexose in the plasma AAT (The results are shown in FIGS. 8 and 9).

The transgenic animals that express rhAAT as described herein were prepared according to the methods described in U.S. Pat. No. 7,045,676, such methods are incorporated herein by reference.

### EQUIVALENTS

The foregoing written specification is considered to be sufficient to enable one skilled in the art to practice the invention. The present invention is not to be limited in scope by examples provided, since the examples are intended as an illustration of certain aspects and embodiments of the invention. Other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A composition comprising alpha-1-antitrypsin (AAT), wherein the AAT is recombinantly produced.

2. The composition of claim 1, wherein the AAT is produced in mammary epithelial cells of a non-human mammal.

3. The composition of claim 1, wherein the AAT is produced in a transgenic non-human mammal.

4. The composition of claim 2 or claim 3, wherein the non-human mammal is a goat, sheep, bison, camel, cow, pig, rabbit, buffalo, horse, rat, mouse or llama.

5. The composition of claim 4, wherein the non-human mammal is a goat.

6. The composition of any one of claims 1-5, wherein the recombinantly produced AAT has enhanced deoxyhexose glycosylation compared to plasma-derived AAT.

7. The composition of any one of claims 1-6, wherein the recombinantly produced AAT has been modified to increase the sialylation on the AAT-glyco-motifs.

8. A composition comprising AAT wherein the AAT has a high level of deoxyhexose glycosylation.

9. A composition comprising AAT wherein the AAT has a high level of sialylation on the AAT-glyco-motifs.

10. A composition comprising AAT wherein the AAT has a high level of deoxyhexose glycosylation and a high level of sialylation on the AAT-glyco-motifs.

11. A composition comprising the AAT of any one of claims 1-10, further comprising milk.

12. A composition comprising the AAT of any one of claims 1-11, further comprising a pharmaceutically acceptable carrier.

13. Mammary gland epithelial cells that produce the AAT of the compositions of any one of claims 1-12.

14. A transgenic non-human mammal comprising the mammary gland epithelial cells of claim 13.

15. A method comprising administering the composition of any one of claims 1-12 to a subject in need thereof.

16. The method of claim 15, wherein the subject has alpha-1-antitrypsin deficiency.

17. The method of claim 15, wherein the subject has an inflammatory disorder.

18. The method of claim 17, wherein the inflammatory disorder is emphysema.

19. The method of any one of claims 15-18, wherein the composition is administered at a dose of from 30 mg/kg to about 60 mg/kg AAT.

20. The method of any one of claims 15-19, wherein the composition is administered intravenously.

21. The method of any one of claims 15-19, wherein the composition is administered by inhalation.

22. A method of reducing elastase activity in the lung, the method comprising administering the composition of any one of claims 1-12 to a subject in an amount sufficient to reduce elastase activity in the lung.
